(19) United States
(12) Patent Application Publication
(10) Pub. No.: US 2013/0204249 A1
(43) Pub. Date: Aug. 8, 2013

(54) INTERNAL ASYMMETRICALLY DESIGNED 8-SHAPED STEEL PLATE FOR TEMPORARY HEMIEPIPHYSIODESIS AND APPLICATION THEREOF

(71) Applicants: Li Zhao, Shanghai (CN); Hai Li, Shanghai (CN); Hongliu Yu, Shanghai (CN)

(72) Inventors: Li Zhao, Shanghai (CN); Hai Li, Shanghai (CN); Hongliu Yu, Shanghai (CN)

(73) Assignee: XIN HUA HOSPITAL AFFILIATED TO SHANGHAI JIAO TONG UNIVERSITY SCHOOL OF MEDICINE, Shanghai (CN)

(21) Appl. No.: 13/879,165

(22) PCT Filed: Nov. 12, 2010
(86) PCT No.: PCT/CN2010/078660

§ 371 Date: Apr. 12, 2013

(30) Foreign Application Priority Data
Oct. 15, 2010 (CN) 201010507530.7

(51) Int. Cl. A61B 17/68

(52) U.S. Cl. CPC A61B 17/68
USPC 606/60

(57) ABSTRACT

An internal asymmetrically designed 8-shaped steel plate (1) for temporary hemiepiphysiodesis includes two single orifice plates (11, 12). Each unilateral orifice plate (11, 12) is provided with a hinged opening (111,121). The two single orifice plates (11, 12) are connected by passing a pivot through the hinged openings (111, 121). The two single orifice plates (11, 12) can be assembled into the asymmetrically 8-shaped steel plate (1). The asymmetrically 8-shaped steel plate (1) is supplied with various specifications and can be provided with various combinations according to the irregular deformity condition on the two sides of patient's physeal line (5).

### TECHNICAL FIELD

The present invention relates to a medical instrument, specifically to an internal asymmetrically 8-shaped steel plate for temporary hemiepiphysiodesis used in the treatment of teenage knee joint angular deformity and application thereof.

### BACKGROUND ART

Limb periarticular angular deformity is a common disease of abnormal lower limbs development, generally including coronal plane angular deformity and sagittal plane angular deformity, the two can exist at the same time. The coronal plane angular deformity mainly includes idiopathic genu varum and genu valgum, Blount disease and other epiphyseal diseases, genu varum and genu valgum caused by wound or tumor. The sagittal plane angular deformity generally shows as knee-joint fixed in flexed position, often secondary to brain paralysis, spina bifida and other neuromuscular system diseases. The clinical manifestations of knee joint angular deformity are abnormal gait, knee-joint pain and dysfunction, severe cases can cause hip-joint and ankle anomaly. Most diseases can be self-healing in childhood, however, pathological lower limbs angular deformity can cause more serious dysfunction if not treated in time, the treatments of pathological lower limbs angular deformity and nonmalignant genu varum and genu valgum are fundamentally different. The angle of thighbone and shinbone of the lower limbs in children has been changed from neonates, children to youngsters. The thighbone and shinbone at an angle of 15 degrees is considered as normal in neonatal period. The lower limbs gradually become straight around 18 months, and thereafter appear slight genu valgum because of posture and activity factors. From then on, under the body's own correction, slight genu valgum is a normal phenomenon when the body reaches maturity, FAT is generally in the range of 4 degrees to 6 degrees. The fundamental difference of physiological and pathological is that the correction procedure of the former is progressive and asymptomatic. The children's lower limbs deformity can be caused by wound, infection, gene, tumor, metabolism, outside force factors and so on. The genu varum and genu valgum with unknown etiology is named idiopathic.

In recent decades, the therapeutic methods for these diseases continue to evolve. The initial therapeutic method is that the patient is treated with cuneiform osteotomy orthopedics of thighbone or shinbone when the children's growth and development basically finish. This method is mainly applied for the epiphyseal plate closure or the correction of adult patient's knee joint angular deformity at present. In 1949, Blount has proposed that the correction of the imbalance development on two sides of epiphyseal plate could be carried out using epiphyseal nail to limit the growth of one side of epiphyseal plate, which created the first on treatment of the knee joint angular deformity by half-epiphyseal block method. Since then the epiphyseal nail is widely applied in treatment of kinds of knee-joint angular deformities, but because of a series of frequent complications such as epiphyseal nail shifting and breakage, the corrective effect is dissatisfactory and thus result in being limited in clinical application. In 1998, Metaizeau et al. proposed that the epiphyseal screw could solve the problem of epiphyseal nail easy to shifting and breakage, but this method the screw directly pass though the epiphyseal plate could cause irreversible damage to epiphyseal plate, its application is restricted to youngsters whose age is close to the maturation of bone age, so its application is in a narrow range. The 8-shaped steel plate for temporary hemiepiphysiodesis is also used in domestic correlative surgery, but the present 8-shaped steel plate is straight plate which could oppress epiphysis and bring about injury after implanted in the body.

China Application of publication No. CN 201316311 Y and publication date Sep. 30, 2009 discloses a U-shaped fastening nail for orthopedic surgery. This utility model discloses a U-shaped fastening nail for orthopedic surgery. It consists of two acuminate titanium alloy nail legs and a piece of curve titanium alloy connector, with an inverted U-shaped as a whole; it is applied in the fixation of epiphyseal plate, bone joint deformity and wound surgery. However, the assembling asymmetrically 8-shaped steel plate has not been reported at present.

### SUMMARY OF THE INVENTION

The purpose of this invention is aimed at overcoming the drawbacks of the prior art and to provide an internal asymmetrically 8-shaped steel plate for temporary hemiepiphysiodesis.

Another purpose of this invention is to provide the use of the internal asymmetrically 8-shaped steel plate for temporary hemiepiphysiodesis.

To achieve the above first purpose, this invention takes the following technical solutions:

An internal asymmetrically 8-shaped steel plate for temporary hemiepiphysiodesis includes two single orifice plates, each said unilateral orifice plate is provided with a hinged opening, the two single orifice plates are connected by passing a pivot through the hinged openings, said two single orifice plates can be assembled into the asymmetrically 8-shaped steel plate.

Said pivot is fixed by a screw and a nut.

Said pivot is fixed by a screw and a clamp spring.

The distant between the centre of the orifice in said unilateral orifice plate and the pivot axis is 6-10 mm.

The distant between the centre of the orifice in said unilateral orifice plate and the pivot axis is 6 mm, 8 mm or 10 mm.

The two distances between the centre of the unilateral orifice and the pivot axis of said one asymmetrically 8-shaped steel plate are different. The two distances between the centre of the unilateral orifice and the pivot axis of said asymmetrically 8-shaped steel plate are 6 mm and 8 mm, or 8 mm and 10 mm, or 6 mm and 10 mm.

There is a gap under each hinged opening of said each unilateral orifice plate, the two gaps could form a non-pressure angle after connecting said two single orifice plates.

To achieve the above second purpose, this invention takes the following technical solutions:

The asymmetrically 8-shaped steel plate is applied as a medical instrument in the disease of knee-joint angular deformity.

The advantages of this invention are:

1. The asymmetrically 8-shaped steel plate is supplied with various specifications and can be provided with various combinations according to the asymmetrically deformity condition on the two sides of patient's physeal line.

2. The unilateral orifice plates of the asymmetrically 8-shaped steel plate are connected together in an articulated way so that the set screw can not bring about an outward cutting force on account of the arc structure of bones, and maintain the steel plate implanted position and the epiphyseal blocking effect.

3. There is a large enough gap under the junction of the two single orifice plates, namely a non-pressure angle, the implanted asymmetrically 8-shaped steel plate couldn't touch the epiphysis and cause epiphyseal oppression to decrease the injuries.

4. As proved by animal experimental researches, the asymmetrically 8-shaped steel plate has an effective control function on the growth and shape of immature epiphysis, and it is a safe and effective medical instrument for half-epiphyseal block surgery.

### BRIEF DESCRIPTION OF THE DRAWINGS

FIG. 1 is a schematic view of the 8-shaped steel plate fixed by a screw and a nut.

FIG. 2 is a schematic view of the 8-shaped steel plate fixed by a groove screw and a clamp spring.

FIG. 3 is a left schematic view of the fully assembled 8-shaped steel plate.

FIG. 4 is a schematic view of the symmetrical 8-shaped steel plate, the two distances between the centre of the unilateral orifice and the pivot axis are 6 mm and 6 mm.

FIG. 5 is a schematic view of the asymmetrically 8-shaped steel plate, the two distances between the centre of the unilateral orifice and the pivot axis are 6 mm and 8 mm.

FIG. 6 is a schematic view of the 8-shaped template of the 8-shaped steel plate surgical tools.

FIG. 7 is a schematic view of the template clamp of the 8-shaped steel plate surgical tools.

FIG. 8 is a schematic view of the working state diagram of the template clamp of the 8-shaped steel plate surgical tools.

FIG. 9 is a schematic view of the angle adjustable double aperture guide apparatus of the 8-shaped steel plate surgical tools.

FIG. 10 is a schematic view of the angle adjusting device of the 8-shaped steel plate surgical tools.

FIG. 11 is a local amplificatory schematic view of the A region in FIG. 9.

FIG. 12 is a schematic view of the working state diagram of the fixed 8-shaped steel plate.

### DETAILED DESCRIPTION OF THE PREFERRED EMBODIMENTS

The embodiments of the invention are further described in detail by combining with figures.

The relevant signs and components of the figures are listed as follows:

| 1. | 8-shaped steel plate | 11. | first unilateral orifice plate |
|---|---|---|---|
| 12. | second unilateral orifice plate | 111. | first hinged opening |
| 121. | second hinged opening | 13. | screw |
| 131. | thread | 14. | nut |
| 15. | groove screw | 151. | groove |
| 16. | clamp spring | 17. | non-pressure angle |
| 112. | first unilateral orifice plate gap | | |
| 122. | second unilateral orifice plate gap | | |
| 2. | 8-shaped template | 21. | first template orifice plate |
| 22. | second template orifice plate | 221. | locating pinhole |
| 222. | template groove | 23. | template pivot |
| 3. | template clamp | 31. | clamp head |
| 311. | head protuberance | 32. | clamp handle |
| 4. | angle adjustable double aperture guide apparatus | | |
| 41. | angle adjusting device | 411. | adjustable angle knob |
| 4111. | locating pin channel | 412. | scale plate |
| 413. | angle pointer | 414. | rotating shaft |
| 415. | attachment screw | | |
| 416. | link block of the double aperture guide apparatus | | |
| 417. | fastening bolt | 42. | pilot sleeve |
| 421. | oriented pore passage | 43. | connecting device |
| 44. | locating pin | 5. | physeal line |

| | |
|---|---|
| 51. | one side of the physeal line |
| 52. | another side of the physeal line |

### EXAMPLE 1

The 8-Shaped Steel Plate Fixed by a Screw and a Nut

As shown in FIG. 1, FIG. 1 is a schematic view of the 8-shaped steel plate fixed by a screw and a nut. Said 8-shaped steel plate (1) includes a first unilateral orifice plate (11), a second unilateral orifice plate (12), a screw (13) and a nut (14). Said first unilateral orifice plate (11) and second unilateral orifice plate (12) are respectively provided with a first hinged opening (111) and a second hinged opening (121). The head of said screw (13) is provided with thread (131), the nut (14) is circular. The two single orifice plates (11, 12) are connected by the screw (13) and the nut (14) through the first hinged opening (111) and the second hinged opening (121). The first hinged opening (111) and the second hinged opening (121) are keeping at coaxial position when assembling, and then the screw (13) is inserted into the two hinged openings, twist the nut (14) along the thread (131), the 8-shaped steel plate (1) is fully assembled.

### EXAMPLE 2

The 8-Shaped Steel Plate Fixed Through a Screw and a Clamp Spring

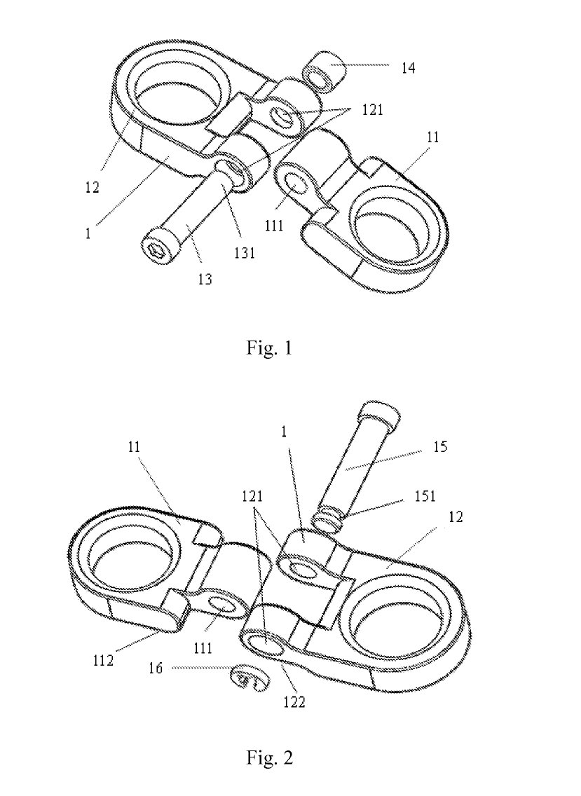

As shown in FIG. 2, FIG. 2 is a schematic view of the 8-shaped steel plate fixed by a groove screw and a clamp spring. Said 8-shaped steel plate (1) includes a first unilateral orifice plate (11), a second unilateral orifice plate (12), a groove screw (15) and a clamp spring (16). Said first unilateral orifice plate (11) and second unilateral orifice plate (12) are respectively provided with a first hinged opening (111) and a second hinged opening (121). The head of said groove screw (15) is provided with a groove (151). The two single orifice plates (11, 12) are connected by the groove screw (15) and the clamp spring (16) through the first hinged opening (111) and the second hinged opening (121). The first hinged opening (111) and the second hinged opening (121) are keeping at coaxial position when assembling, and then the groove screw (15) is inserted into the two hinged openings, add the clamp spring (16) on the groove (151), the 8-shaped steel plate (1) is fully assembled.

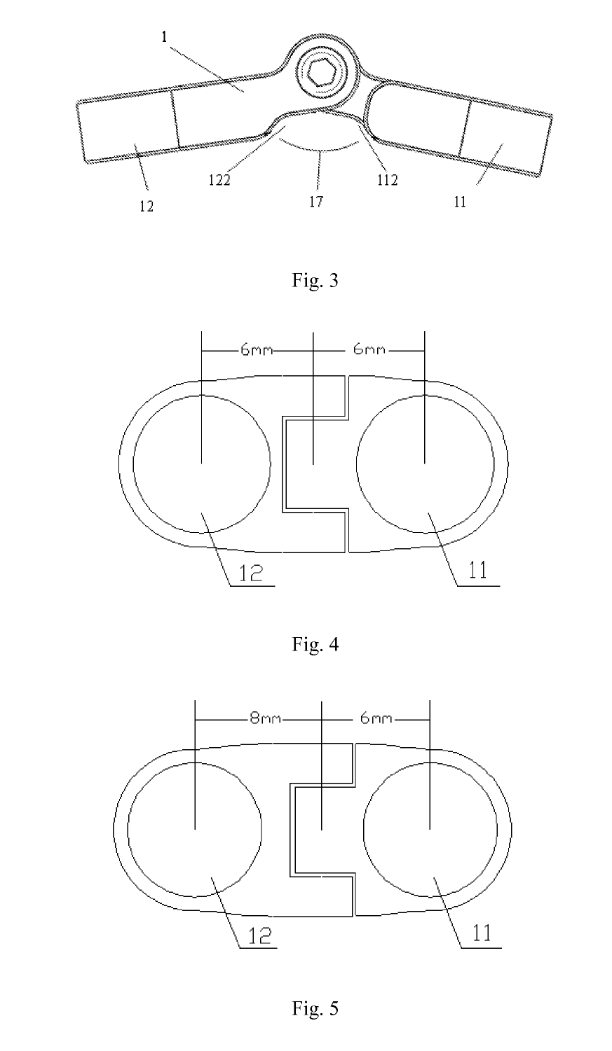

It should be noted that there are a first unilateral orifice plate gap (112) under the first hinged opening (111) of the first unilateral orifice plate (11) and a second unilateral orifice plate gap (122) under the second hinged opening (121) of the second unilateral orifice plate (12). The first unilateral orifice plate gap (112) and the second unilateral orifice plate gap (122) could form an arc-shaped gap after the 8-shaped steel plate (1) is fully assembled, named non-pressure angle (17). As shown in FIG. 3, FIG. 3 is a left schematic view of the fully assembled 8-shaped steel plate (1). The non-pressure angle (17) provides a large enough gap under the hinge of the 8-shaped steel plate (1), the implanted 8-shaped steel plate couldn't touch the epiphysis and cause epiphyseal oppression to decrease the injuries.

### EXAMPLE 3

The 8-Shaped Steel Plate Provided with Different Size Combination

Both the first unilateral orifice plate (11) and the second unilateral orifice plate (12) of the 8-shaped steel plate (1) comprise three kinds of specifications; the distant between the centre of the orifice in said unilateral orifice plate and the pivot axis is 6 mm, 8 mm or 10 mm. These orifice plates can be assembled into six kinds of different 8-shaped steel plates. According to the actual sizes of the two sides of patient's epiphysis, doctors can select different pair-combination from six kinds of combinations.

(1) The symmetrical 8-shaped steel plate, the two distances between the centre of the unilateral orifice and the pivot axis are 6 mm and 6 mm As shown in FIG. 4, FIG. 4 is a schematic view of the symmetrical 8-shaped steel plate, the two distances between the centre of the unilateral orifice and the pivot axis are 6 mm and 6 mm. The first unilateral orifice plate (11) of 6 mm and the second unilateral orifice plate (12) of 6 mm are selected and connected according to the installation method in example 1, the two single orifice plates can be assembled into the symmetrical 8-shaped steel plate with 6 mm-6 mm combination.

(2) The symmetrical 8-shaped steel plate provided, the two distances between the centre of the unilateral orifice and the pivot axis are 8 mm and 8 mm.

The first unilateral orifice plate (11) of 8 mm and the second unilateral orifice plate (12) of 8 mm are selected and connected according to the installation method in example 1, the two single orifice plates can be assembled into the symmetrical 8-shaped steel plate with 8 mm-8 mm combination.

(3) The symmetrical 8-shaped steel plate, the two distances between the centre of the unilateral orifice and the pivot axis are 10 mm and 10 mm. The first unilateral orifice plate (11) of 10 mm and the second unilateral orifice plate (12) of 10 mm are selected and connected according to the installation method in example 1, the two single orifice plates can be assembled into the symmetrical 8-shaped steel plate with 10 mm-10 mm combination.

(4) The asymmetrically 8-shaped steel plate, the two distances between the centre of the unilateral orifice and the pivot axis are 6 mm and 8 mm. As shown in FIG. 5, FIG. 5 is a schematic view of the asymmetrically 8-shaped steel plate, the two distances between the centre of the unilateral orifice and the pivot axis are 6 mm and 8 mm. The first unilateral orifice plate (11) of 6 mm and the second unilateral orifice plate (12) of 8 mm are selected and connected according to the installation method in example 1, the two single orifice plates can be assembled into the asymmetrically 8-shaped steel plate with 6 mm-8 mm combination.

(5) The asymmetrically 8-shaped steel plate, the two distances between the centre of the unilateral orifice and the pivot axis are 6 mm and 10 mm. The first unilateral orifice plate (11) of 6 mm and the second unilateral orifice plate (12) of 10 mm are selected and connected according to the installation method in example 1, the two single orifice plates can be assembled into the asymmetrically 8-shaped steel plate with 6 mm-10 mm combination.

(6) The asymmetrically 8-shaped steel plate, the two distances between the centre of the unilateral orifice and the pivot axis are 8 mm and 10 mm. The first unilateral orifice plate (11) of 8 mm and the second unilateral orifice plate (12) of 10 mm are selected and connected according to the installation method in example 1, the two single orifice plates can be assembled into the asymmetrically 8-shaped steel plate with 8 mm-10 mm combination.

### EXAMPLE 4

The Surgical Tools of the 8-Shaped Steel Plate

The surgical tools of the internal 8-shaped steel plate for temporary hemiepiphysiodesis are designed in accordance with the sizes of the 8-shaped steel plate (1) and the 8-shaped template (2), which can ensure the accurate and fast implantation of the 8-shaped steel plate (1) and decrease the injuries for patient's epiphysis.

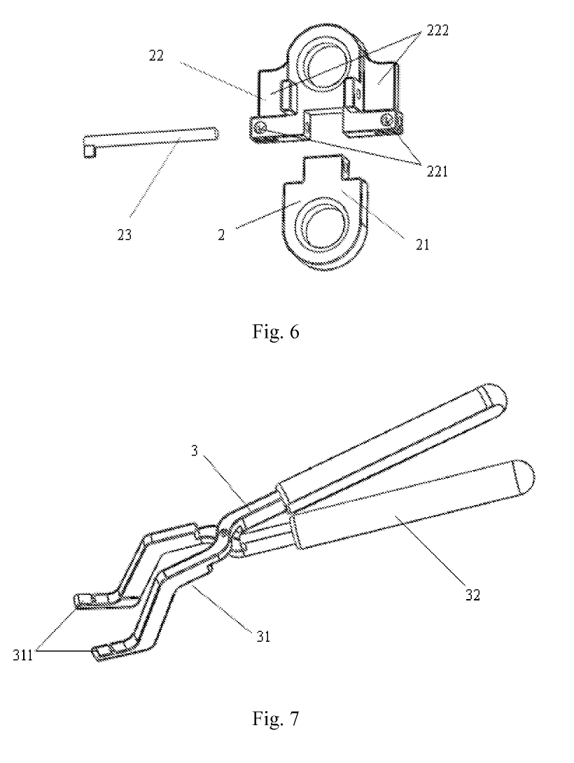

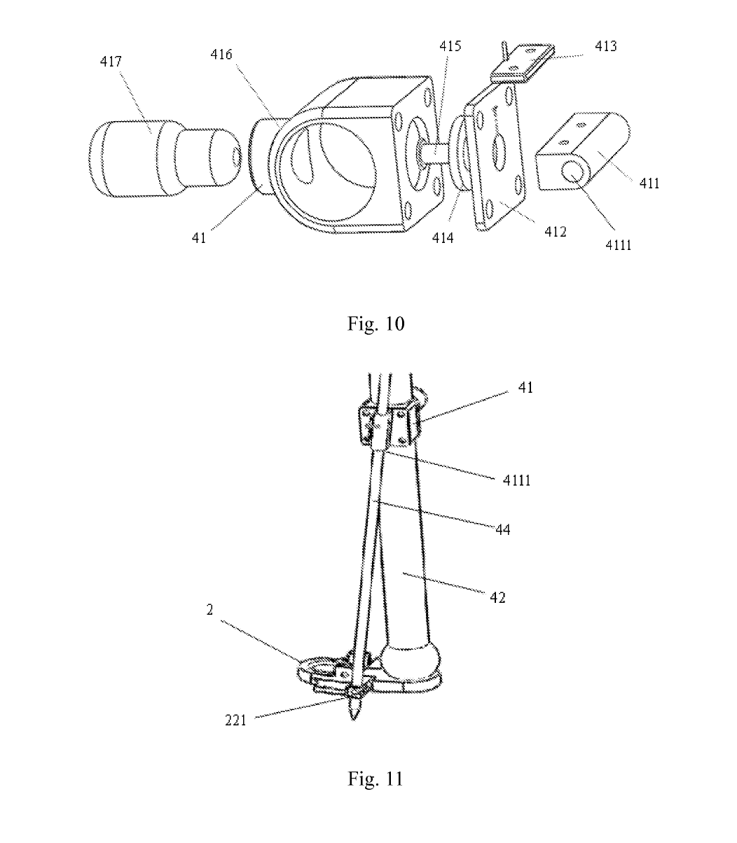

Said surgical tools include: an 8-shaped template (FIG. 6), a template clamp (FIG. 7) and an angle adjustable double aperture guide apparatus (FIG. 9), said angle adjustable double aperture guide apparatus is provided with an angle adjusting device (FIG. 10).

(1) 8-Shaped Template (2)

As shown in FIG. 6, FIG. 6 is a schematic view of the 8-shaped template of the 8-shaped steel plate surgical tools. The size of the 8-shaped template (2) is same as that of the 8-shaped steel plate (1); the 8-shaped template (2) can also be disassembled and assembled, and includes a first template orifice plate (21), a second template orifice plate (22) and a template pivot (23). There are two locating pinholes (221) on the second template orifice plate (22) and two template grooves (222) on the clamping surface. The installation method of the 8-shaped template (2) is same as that of the 8-shaped steel plate (1); the 8-shaped template (2) can be assembled into symmetrical or asymmetrically template in accordance with the needed 8-shaped steel plate (1) in surgery. The first template orifice plate (21) and the second template orifice plate (22) of the 8-shaped template (2) comprise three kinds of dimensions respectively; the distant between the centre of the orifice in the unilateral orifice plate and the pivot axis is 6 mm, 8 mm or 10 mm. These orifice plates can be assembled into six kinds of different 8-shaped templates (2), including the symmetrical 8-shaped template with 6 mm-6 mm combination, the symmetrical 8-shaped template with 8 mm-8 mm combination, the symmetrical 8-shaped template with 10 mm-10 mm combination, the asymmetrically 8-shaped template with 6 mm-8 mm combination, the asymmetrically 8-shaped template with 6 mm-10 mm combination and the asymmetrically 8-shaped template with 8 mm-10 mm combination. According to the actual sizes of the two sides of patient's epiphysis, doctors can select different pair-combination from six kinds of combinations.

(2) Template Clamp (3)

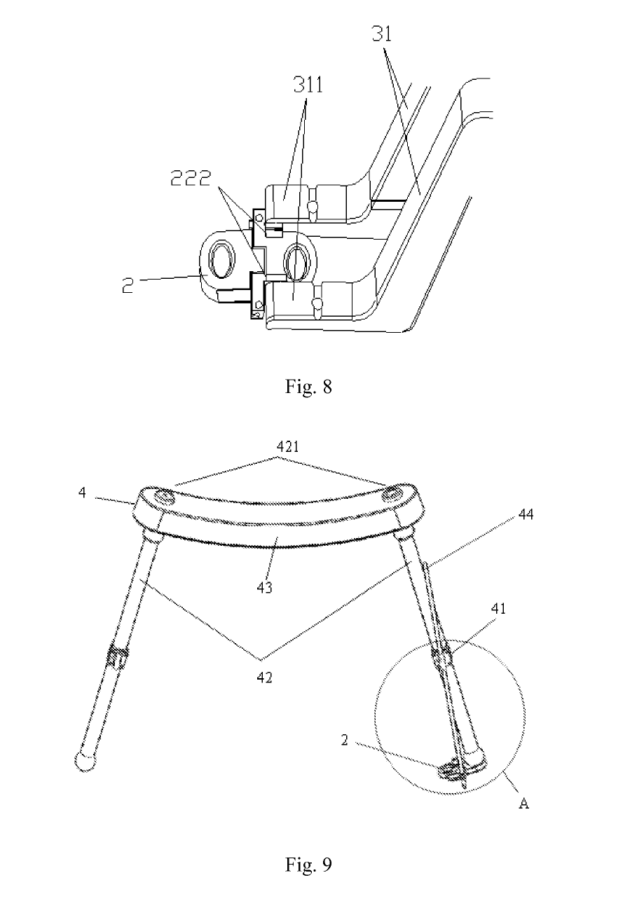

As shown in FIG. 7, FIG. 7 is a schematic view of the template clamp of the 8-shaped steel plate surgical tools. The template clamp (3) includes two clamp heads (31) and two clamp handles (32), each clamp head (31) is provided with a head protuberance (311) at the end. As shown in FIG. 8, FIG. 8 is a schematic view of the working state diagram of the template clamp of the 8-shaped steel plate surgical tools. The two clamp heads (31) are parallel when the template clamp (3) is clenching, and the distance of the two clamp heads (31) is same as that of the two clamping surfaces of the 8-shaped template (2). The two head protuberances (311) of the two clamp heads (31) just touch the two template grooves (222) on the clamping surfaces of the 8-shaped template (2).

(3) Angle Adjustable Double Aperture Guide Apparatus (4)

As shown in FIG. 9, FIG. 9 is a schematic view of the angle adjustable double aperture guide apparatus of the 8-shaped steel plate surgical tools. The angle adjustable double aperture guide apparatus (4) includes an angle adjusting device (41), two pilot sleeves (42), a connecting device (43) and a locating pin (44). Said two pilot sleeves (42) and connecting device (43) together compose an-shaped structure. Each pilot sleeve (42) is fixed with an angle adjusting device (41) and supplied with an oriented pore passage (421) in interior space, the diameters of the two oriented pore passages (421) are different so as to use in different drilling diameters.

In order to avoid damaging the epiphyseal organization and drill accurately, the design use the angle adjusting device (41) to adjust the angle of the center lines of the locating pin (44) and the drill hole. The angle adjusting device (41) is provided with angle index that is easy to adjust to the appropriate angle. As shown in FIG. 10, FIG. 10 is a schematic view of the angle adjusting device of the 8-shaped steel plate surgical tools. Said angle adjusting device (41) is provided with an adjustable angle knob (411), a scale plate (412) and a rotating shaft (414), connected by an attachment screw (415), an angle pointer (413) is fixed on the adjustable angle knob (411) with screws. The connected whole is fixed on the link block of the double aperture guide apparatus (416) through the four screw holes of the scale plate (412), and then fixed on the pilot sleeve (42) by the link block of the double aperture guide apparatus (416). A fastening bolt (417) can make the angle adjusting device (41) fix on the accurate position of the pilot sleeve (42).

As shown in FIG. 11, FIG. 11 is a local amplificatory schematic view of the A region in FIG. 9. Before the operation, a suitable 8-shaped steel plate (1) and a suitable 8-shaped template (2) are selected according to the deformity condition on the two sides of patient's physeal line. The locating pin (44) is inserted into the specific position of the exposed bone after operative incision and the 8-shaped template (2) is placed by the locating pinhole (221). The required angle is adjusted by the angle adjusting device (41) and the pilot sleeve (42) is placed along the locating pin (44) which passes through the locating pin channel (4111) of the angle adjusting device (41). Then punching is carried out using a special drill through the oriented pore passage (421) of the pilot sleeve (42), in the same way, get another drill hole. The 8-shaped steel plate (1) is put into and fixed by screw after getting out the 8-shaped template (2); finally, the operative incision is sutured.

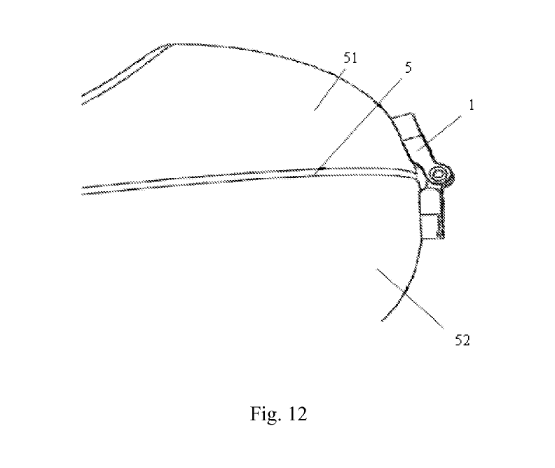

As shown in FIG. 12, FIG. 12 is a schematic view of the working state diagram of the fixed 8-shaped steel plate. It should be noted that the size of the 8-shaped steel plate (1) is determined by the deformity condition of one side of the physeal line (51) and another side of the physeal line (52) of the physeal line (5). In using state, the pivot centre line of the 8-shaped steel plate (1) and the physeal line (5) are in the same plane. This kind of 8-shaped steel plate (1) can further avoid the repeated surgery caused by the fixed dimension which can not be suitable for different actual condition of the patient's bones, and decrease the suffering of patient.

### EXAMPLE 5

The Animal Research of the 8-Shaped Steel Plate for Hemiepiphysiodesis

### (1) MATERIALS AND METHODS

Thirty (sixty knees) New Zealand white rabbits of forty-six weeks are randomly divided into two groups on average. For the first group, the 8-shaped steel plate is implanted from extraperiosteal into the two ends of the epiphysis which is located at the proximal shinbone of one knee, and the sham operation of another knee is carried out as control group. Three weeks later, the rabbits are put to death to get the local pathological organization after taking photos under X-ray. The second group is randomly divided into two subgroups with seven rabbits (A-subgroup) and eight rabbits (B-subgroup). The 8-shaped steel plate is implanted into the two ends of the epiphysis which is located at the proximal shinbone (A-subgroup from periosteal outside, B-subgroup from periosteal inside). Three weeks later, all of 8-shaped steel plates are taken out after taking photos under X-ray; six weeks later, all of the rabbits are put to death to get the local pathological organization after again taking photos under X-ray. All of the rabbits are not required braking after operation.

### (2) RESULTS

All of the rabbits have not show the local adverse reaction after operation, and the activities of the knee-joints have no obvious change.

The first group: the angles of the articular surface diaphysis (ALDA) of the proximal shinbone of the New Zealand white rabbits are 84-88 degrees under X-ray before operation, an average of 86 degrees, after implanting the 8-shaped steel plates, the values of ALDA are reduced to 56-61 degrees, an average of 58 degrees (P<0.05) in three weeks later. The pathological results show that, after implanting the 8-shaped steel plates, the cell proliferation of the growth plate still exist in three weeks later.

The A-subgroup of the second group: the angles of the articular surface diaphysis (ALDA) of the proximal shinbone of the New Zealand white rabbits are 85-87 degrees under X-ray before operation, an average of 85 degrees, after implanting the 8-shaped steel plates, the values of ALDA are reduced to 53-60 degrees, an average of 56 degrees (P<0.05) in three weeks later. The pathological results show that the cell proliferations of the growth plate still exist. The values of ALDA are improved to 65-72 degrees, an average of 68 degrees (P<0.05) in six weeks later. The pathological results show that the cell proliferations of the growth plate still exist.

The B-subgroup of the second group: the angles of the articular surface diaphysis (ALDA) of the proximal shinbone of the New Zealand white rabbits are 84-89 degrees under X-ray before operation, an average of 83 degrees, after implanting the 8-shaped steel plates, the values of ALDA are reduced to 42-49 degrees, an average of 45 degrees (P<0.05) in three weeks later. The pathological results show that the cell proliferations of the growth plate are obviously less than other organization and a small amount of bone bridge form. The values of ALDA are improved to 48-54 degrees, an average of 51 degrees (P<0.05) in six weeks later. The pathological results show that the bone bridge still form and the cell proliferations of the growth plate exist.

### (3) CONCLUSIONS

The periosteum does not need to be stripped when implanting the 8-shaped steel plate; the operation is simple and not required braking post operation. The 8-shaped steel plate has an effective control function on the growth and shape of the immature epiphysis; it is a safe and effective operation method for half-epiphyseal block.

1. An internal asymmetrically 8-shaped steel plate for temporary hemiepiphysiodesis includes two single orifice plates, wherein each said unilateral orifice plate is provided with a hinged opening, the two single orifice plates are connected by passing a pivot through the hinged openings, said two single orifice plates can be assembled into the asymmetrically 8-shaped steel plate.

2. The asymmetrically 8-shaped steel plate according to claim 1, wherein said pivot is fixed by a screw and a nut.

3. The asymmetrically 8-shaped steel plate according to claim 1, wherein said pivot is fixed by a screw and a clamp spring.

4. The asymmetrically 8-shaped steel plate according to any one of claims 1 to 3, wherein the distant between the centre of the orifice in said unilateral orifice plate and the pivot axis is 6-10 mm.

5. The asymmetrically 8-shaped steel plate according to claim 4, wherein the distant between the centre of the orifice in said unilateral orifice plate and the pivot axis is 6 mm, 8 mm or 10 mm.

6. The asymmetrically 8-shaped steel plate according to claim 5, wherein the two distances between the centre of the unilateral orifice and the pivot axis of said one asymmetrically 8-shaped steel plate are different.

7. The asymmetrically 8-shaped steel plate according to claim 6, wherein the two distances between the centre of the unilateral orifice and the pivot axis of said asymmetrically 8-shaped steel plate are 6 mm and 8 mm, or 8 mm and 10 mm, or 6 mm and 10 mm.

8. The asymmetrically 8-shaped steel plate according to claim 7, wherein there is a gap under each hinged opening of said each unilateral orifice plate, the two gaps could form a non-pressure angle after connecting said two single orifice plates.

9. The asymmetrically 8-shaped steel plate according to any one of claims 1 to 8 is applied as a medical instrument in the disease of knee joint angular deformity.
